(19) United States
(12) Patent Application Publication
(10) Pub. No.: US 2013/0072459 A1
(43) Pub. Date: Mar. 21, 2013

(54) PHARMACEUTICAL COMPOSITION FOR THE PREVENTION OR THE TREATMENT OF NON-ALCOHOLIC FATTY LIVER DISEASE AND THE METHOD FOR PREVENTION OR TREATMENT OF NON-ALCOHOLIC FATTY LIVER DISEASE USING THE SAME

(71) Applicants: Gook-Jun An, Gyeonggi-do (KR); Eun-Kyoung Yang, Seoul (KR); Eun-Jung Cho, Seoul (KR); Yu-Na Chae, Gyeonggi-do (KR); Song-Hyen Choi, Gyeonggi-do (KR); Ha-Dong Kim, Incheon (KR); Chang-Yell Shin, Seoul (KR); Mi-Kyung Kim, Gyeonggi-do (KR); Woo-Young Kwak, Gyeonggi-do (KR); Heung-Jae Kim, Gyeonggi-do (KR); Moon-Ho Son, Gyeonggi-do (KR); Soon-Hoe Kim, Gyeonggi-do (KR)

(72) Inventors: Gook-Jun An, Gyeonggi-do (KR); Eun-Kyoung Yang, Seoul (KR); Eun-Jung Cho, Seoul (KR); Yu-Na Chae, Gyeonggi-do (KR); Song-Hyen Choi, Gyeonggi-do (KR); Ha-Dong Kim, Incheon (KR); Chang-Yell Shin, Seoul (KR); Mi-Kyung Kim, Gyeonggi-do (KR); Woo-Young Kwak, Gyeonggi-do (KR); Heung-Jae Kim, Gyeonggi-do (KR); Moon-Ho Son, Gyeonggi-do (KR); Soon-Hoe Kim, Gyeonggi-do (KR)

(21) Appl. No.: 13/636,670

(22) PCT Filed: Mar. 23, 2011
(86) PCT No.: PCT/KR2011/001988

§ 371 Date: Nov. 21, 2012

(30) Foreign Application Priority Data
Mar. 24, 2010 (KR) 1020100026134

(51) Int. Cl. A61K 31/675; A61K 31/4965; A61K 31/522; A61P 1/16; A61P 3/06; A61P 3/10; A61P 3/04; A61K 31/4985; A61K 31/40

(52) U.S. Cl. 514/81; 514/249; 514/255.01; 514/263.21; 514/423

(57) ABSTRACT

The present invention provides a pharmaceutical composition for the prevention and treatment of a non-alcoholic fatty liver disease (NAFLD), containing an active ingredient selected from the group consisting of Compound 1 represented by formula 1, sitagliptin, vildagliptin, linagliptin or a pharmaceutically acceptable salt thereof. Further, the present invention provides a method for the prevention or treatment of a non-alcoholic fatty liver disease, including administering an effective amount of an active ingredient selected from the group consisting of Compound 1 represented by formula 1, sitagliptin, vildagliptin, linagliptin or a pharmaceutically acceptable salt thereof to a mammal including a human in need thereof. Further, the present invention provides use of Compound 1 represented by formula 1, sitagliptin, vildagliptin, linagliptin or a pharmaceutically acceptable salt thereof, for manufacturing a pharmaceutical composition for the prevention or treatment of a non-alcoholic fatty liver disease.

### TECHNICAL FIELD

The present invention relates to a pharmaceutical composition for the prevention or treatment of a non-alcoholic fatty liver disease and a method for the prevention or treatment of a fatty liver disease.

### BACKGROUND ART

Non-alcoholic fatty liver disease (NAFLD) refers to a broad spectrum of diseases including simple steatosis which is accompanied by no inflammatory response in a patient with no excessive intake of alcohol, and non-alcoholic steatohepatitis (NASH), liver fibrosis and liver cirrhosis which result from the progression of simple steatosis and exhibit hepatocellular inflammation and is a broader concept as compared to the former term non-alcoholic steatohepatitis (Ludwig J et al., Mayo Clin Proc 1980, 55(7):434-438).

NAFLDs may be categorized into a primary NAFLD and a secondary NAFLD depending on the pathological cause. It is known that the primary one is caused by hyperlipidemia, diabetes, obesity or the like which is a characteristic of metabolic syndrome, whereas the secondary one is caused by nutritional causes (sudden body weight loss, starvation, intestinal bypass surgery), various drugs (glucocorticoids, estrogens, tamoxifen, methotrexate, zidovudine, amiodarone, tetracycline, didanosine, cocaine, diltiazem, perhexyline), toxic substances (poisonous mushrooms, bacterial toxins), metabolic causes (lipodystrophy, dysbetalipoproteinemia, Weber-Christian syndrome, Wolman's disease, acute fatty liver of pregnancy, Reye's syndrome) and other factors (inflammatory bowel syndrome, AIDS infection: HIV infection) (Adams L A et al., CMAJ 2005, 172(7):899-905). It is known that the incidence of NAFLDs linked to diabetes and obesity which are important characteristics of metabolic syndrome that is a primary factor is about 50% of diabetic patients, about 76% of obesity patients, and most of obese diabetic patients (Gupte P et al., J Gastroenterol Hepatol 2004, 19(8):854-858). Further, when a liver biopsy is made in diabetic and obesity patients with an increased level of alanine aminotransferase (ALT), the incidence of steatohepatitis is in the range of 18 to 36% (Braillon A et al., Gut 1985, 26(2):133-139), and it is known that steatohepatitis is caused by insulin resistance. Further, the proportion of steatohepatitis being progressed into liver cirrhosis in steatohepatitis patients who also have diabetes, obesity and hyperlipidemia varies depending on the investigation period of the disease. It was reported that the proportion of patients with the progression of steatohepatitis into liver cirrhosis within the investigation period of 3 to 11 years is in the range of 4 to 26%, and the lethality of such patients is high as compared to a general population group (Powell E E et al., Hepatology 1990, 11(1):74-80, Bacon B R et al., Gastroenterology 1994, 107(4):1103-1109, Matteoni C A et al., Gastroenterology 1999, 116(6):1413-1419).

It is known that hepatic accumulation of triglyceride which is directly linked to

NAFLDs and consequent hepatocellular damage may appear due to an imbalance between hepatic inflow/synthesis and release/oxidation of lipids due to changes in systemic factors such as local factors and insulin resistance. That is, it is known that hepatic accumulation of triglyceride results from a hepatic inflow of fatty acid at a level higher than the capacity of hepatocytic mitochondria to oxidize fatty acid, due to hyperinsulinemia resulting from insulin resistance (Reid A E. Gastroenterology 2001, 121(3): 710-723). Further, insulin resistance leads to an increased expression (up-regulation) of peroxisome proliferator-activated receptor-gamma (PPAR-γ) and sterol regulatory element binding protein-1c (SREBP-1c) that are lipogenic transcription factors, which may result in the accumulation of triglyceride due to increased de novo hepatic lipogenesis (Fromenty B et al., Diabetes Metab 2004, 30(2):121-138).

Fat is released in the form of very low density lipoprotein (VLDL) into the blood from the liver. Meanwhile, VLDL is formed by a microsomal triglyceride transfer protein (MTP) which binds triglyceride to apolipoprotein B (apo B). Insulin resistance results in increased decomposition of fat in adipose tissues, thereby leading to an increase in blood fatty acid. Consequent decreases in the activity of MTP and the synthesis of apo B lead to decreased hepatic release of fat and accumulation of triglyceride (Namikawa C et al., J Hepatol 2004, 40(5):781-786). The reason why insulin resistance is particularly important for the pathogenesis of NAFLDs is because there is a high interrelationship between the metabolic syndrome characterized by diabetes, obesity and hyperlipidemia and the non-alcoholic fatty liver. The fat-accumulated liver is susceptible to secondary damage and therefore progresses into hepatocytic inflammation and fibrosis.

Such secondary damage is caused by various adipocytokines including tumor necrosis factor-alpha (TNF-α), leptin and adiponectin, oxidative stress, lipid peroxidation, increased fatty acid (Hui J M et al., Hepatology 2004, 40(1):46-54), and gut-derived bacterial endotoxins in patients who had a jejunoileal bypass surgery (Day C P and James O F. Gastroenterology 1998, 114(4):842-845).

A microvascular flow is inhibited by hepatocytes with deposition of lipid droplets due to the progression of hepatic damage and perisinusoidal fibrosis, which consequently leads to a decrease in exchange of oxygen and nutritive substances and the occurrence of microvascular inflammatory responses (Magalotti D et al., Dig Liver Dis 2004, 36(6):406-411). Further, steatohepatitis patients exhibit increased blood levels of ferritin and iron ions, and increased levels of iron ions, tumor growth factor-β1 (TGF-β1) and cytokines result in the activation of hepatic stellate cells and the synthesis of collagen, thus leading to the progression of liver fibrosis and liver cirrhosis (Pietrangelo A et al., Hepatology 1994, 19(3):714-721).

Meanwhile, it has recently been reported that NAFLDs are associated with cardiovascular diseases (CVDs) including atherosclerosis, cerebrovascular diseases (Francazani A et al., Am J Med 2008, 121:72-78), microvascular diseases, nephropathy, and retinopathy (Targher G et al., Diabetologia 2008; 51(3):444-450), polycystic ovarian syndrome (PCOS) (Targher G et al., Atherosclerosis 2007, 191:235-240, Cerda C et al., J Hepatol 2007, 47:412-417), or obstructive sleep anpea (OSA) (Tanne F et al., Hepatology 2005, 41:1290-1296).

There is no established method for NAFLDs up to now. This is because the incidence of NAFLDs is associated with a variety of factors such as diabetes, obesity, coronary artery diseases, and sitting habits. Obesity is an important target in the treatment of NAFLDs, since the reduction of body weight may lead to decreases in factors associated with insulin resistance which is a risk factor of hepatic damage, an inflow amount of fatty acid into the liver, and inflammatory or fibrotic adipokines. Although alanine aminotransferase (ALT) levels and hepatic triglyceride content may be lowered due to the reduction of body weight through dietary control and physical exercise, amelioration of ALT level and hepatic triglyceride content by the reduction of body weight is little known in patients with necrotic inflammation or liver fibrosis (Harrison S A et al., Gut 2007, 56:1760-1769). The intake of dietary saturated fat is highly linked to hepatic triglyceride content and insulin resistance (Westerbacka J et al., J Clin Endocrinol Metab 2005, 2804-2809), and therefore dietary control is very important. Physical exercise for the reduction of body weight and the amelioration of insulin resistance is known to provide histological amelioration of fatty liver (Ueno T et al., J Hepatol 1997, 27:103-110).

There is a report that orlistat, which is an intestinal lipase inhibitor and is used as an oral anti-obesity drug, exhibits histological improvements of the liver in patients with steatohepatitis (Hussein O et al., Dig Dis Sci 2007, 52:2512-2519). However, it is not clear that such histological improvements are attributable to the reduction of body weight or other mechanisms.

Type 2 diabetes and insulin resistance are known to be involved in inflammation and fibrosis of the liver (Adachi M et al., Gastroenterology 2007, 132:1434-1446). When metformin is administered to type 2 diabetic patients with manifestation of non-alcoholic fatty liver diseases, amelioration of fatty liver was not clear with the hematological examination and nuclear magnetic resonance imaging. However, there is a report that one-year administration of metformin exhibited decreases in blood levels of hepatic enzymes and hepatic necrotic inflammation and fibrosis in NAFLD patients with no exhibition of diabetes, as compared to the group to which vitamin E or a body weight-decreasing drug is administered (Bugianesi E et al., Am J Gastroenterol 2005, 100:1082-1090).

Thiazolidinedione (TZD) class drugs are PPAR-γ agonists, improve insulin sensitivity, inhibit the accumulation of fat in the liver and muscles, and increase the secretion of adipokines having anti-inflammatory and anti-fibrotic actions in adipocytes. TZD class drugs have been reportedly to exhibit direct anti-fibrotic actions on the liver in animal models of non-alcoholic fatty liver diseases (Galli A et al., Gastroenterology 2002, 122:1924-1940).

The second-generation TZD, pioglitazone, has been reported to exhibit amelioration of fatty liver and significant amelioration of inflammatory and necrotic responses in steatohepatitis patients (Belfort R et al., N Engl J Med 2006, 355:2297-2307), but has disadvantages associated with aggravation of fatty liver and inflammation when administration of the drug is stopped in steatohepatitis patients (Lutchman G et al., Hepatology 2007, 46:424-429).

Dyslipidemia is associated with non-alcoholic fatty liver diseases. Hypertriglyceridemia appears in 20 to 80% of non-alcoholic fatty liver patients and fibrate class drugs, which are blood triglyceride-lowering drugs, may be therapeutically beneficial for hypertriglyceridemia. Fibrate class drugs are PPAR-α receptor agonists and their medicinal efficacy was investigated in animal models of steatohepatitis (Ip E et al., Hepatology 2004, 39:1286-1296). Unfortunately, it was reported that clofibrate, which is one of fibrate class drugs, has no effects on hepatic enzyme levels and histological lesions in a clinical testing (Laurin J et al., Hepatology 1996, 23:1464-1467).

In the case of statin class drugs which have the mechanism of 3-hydroxy-3-methyl-glutaryl-CoA reductase inhibitor (HMG CoA reductase inhibitor) that is a cholesterol-lowering substance, although their effects in NAFLD patients have not yet been established, these statin class drugs have an advantage of safe prescription to type 2 diabetic patients and patients with risk factors of cardiovascular diseases. However, hepatotoxicity of statin class drugs may disadvantageously lead to an increased blood level of alanine aminotransferase (Browning J et al., Hepatology 2006, 44:466-471).

In the case of anti-hypertensive agents, alpha-blockers exhibited medicinal efficacy in animal models showing liver fibrosis and steatohepatitis (Hirose A et al., Hepatology 2007, 45:1375-1381) and displayed a decrease in serum factors of liver fibrosis and an insulin sensitizing effect in steatohepatitis patients under clinical testing, thus presenting therapeutic potentialities thereof (Ichikawa Y et al., Intern Med 2007, 46:1331-1336).

### DISCLOSURE OF INVENTION

### Technical Problem

Therefore, the present invention is intended to provide a pharmaceutical composition for the prevention or treatment of a non-alcoholic fatty liver disease (NAFLD) and a method for the treatment of a NAFLD using the same.

### Solution to Problem

The present invention relates to a pharmaceutical composition for the prevention or treatment of a non-alcoholic fatty liver disease, comprising an active ingredient selected from the group consisting of compounds represented by the following formula 1, 2, 3 or 4 or a pharmaceutically acceptable salt thereof.

Compound 1 represented by formula 1 is ((R)-4-[(R)-3-amino-4-(2,4,5-trifluorophenyl)-butanoyl]-3-(t-butoxymethyl)piperazin-2-one) and is disclosed in Korean Patent Application No. 2008-0036052.

Compound 2 represented by formula 2 is sitagliptin and is commercially available under the trade name of Januvia; Compound 3 represented by formula 3 is vildagliptin and is commercially available under the trade name of Galvus; and Compound 4 represented by formula 4 is known as linagliptin.

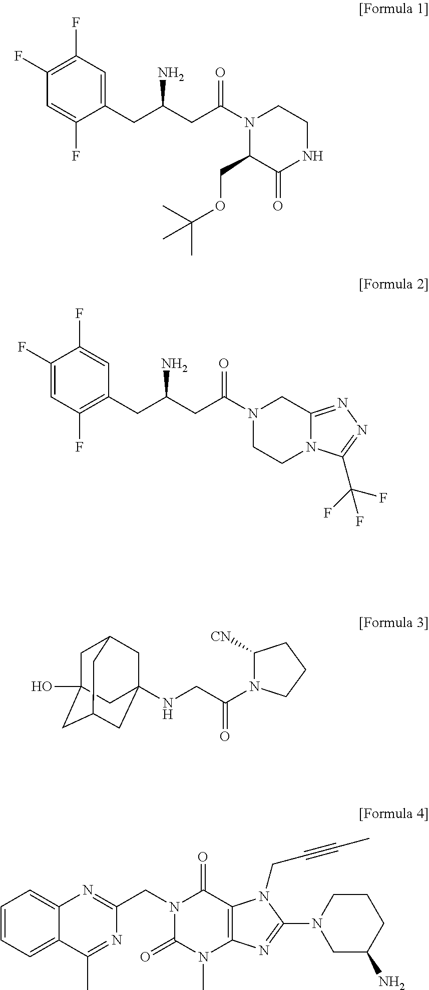

Compounds 1 to 4 in accordance with the present invention may be synthesized by a conventionally known method or are commercially available (for example, vildagliptin is commercially available from Trademax, China).

As shown in formula 1 or 2 above, Compound 1 or 2 may have asymmetric centers at the beta carbon and at the carbon of the 3-position of the piperazinone ring. Single enantiomers, single diastereoisomers, racemates, or mixtures of diastereoisomers may fall within the scope of Compound 1 or 2 of formula 1 or 2 in accordance with the present invention. Further, Compound 1 or 2 of formula 1 or 2 in accordance with the present invention may be in the form of a tautomer, and individual tautomers as well as mixtures thereof may fall within the scope of Compound 1 or 2 in accordance with the present invention. The beta amino group-containing hetero compound of Compound 1 or 2 in accordance with the present invention includes a pharmaceutically acceptable salt thereof, as well as a hydrate and solvate that may be prepared therefrom. A pharmaceutically acceptable salt of the beta amino group-containing hetero compound of Compound 1 or 2 may be prepared by any conventional method for the preparation of salts known in the art. Further, Compounds 3 and 4 include all of possible optical isomers and pharmaceutically acceptable salts thereof.

As used herein, the term “pharmaceutically acceptable salt” refers to a salt prepared from a pharmaceutically acceptable non-toxic base or acid, including an inorganic or organic base and an inorganic or organic acid. Examples of the salt derived from an inorganic base include salts with aluminum, ammonium, calcium, copper, iron (I), iron (II), lithium, magnesium, manganese, potassium, sodium and zinc. In particular, an ammonium, calcium, magnesium, potassium or sodium salt is preferable. A solid salt may be present in the form of one or more crystal structures or in the form of a hydrate. Examples of the salt derived from a pharmaceutically acceptable non-toxic organic base include salts of primary, secondary, and tertiary amines, substituted amines including naturally occurring substituted amines, cyclic amines, and basic ion exchange resins, such as arginine, betaine, caffeine, choline, N′,N′-dibenzylethylenediamine, diethylamine, 2-diethylaminoethanol, 2-dimethylaminoethanol, ethanolamine, ethylendiamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, and tromethamine.

When the compound of the present invention is basic, a salt thereof may be prepared from a pharmaceutically acceptable non-toxic acid including an inorganic or organic acid. Examples of the acid include acetic acid, benzenesulfonic acid, benzoic acid, camphorsulfonic acid, citric acid, ethanesulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid, and p-toluenesulfonic acid. Citric acid, hydrobromic acid, hydrochloric acid, maleic acid, phosphoric acid, sulfuric acid, fumaric acid or tartaric acid is preferable.

The hydrate of Compound 1, 2, 3 or 4 of the present invention or a pharmaceutically acceptable salt thereof may be understood to contain a stoichiometric or non-stoichiometric amount of water bound via non-covalent intermolecular forces. The hydrate may contain more than 1 equivalent of water, typically 1 to 5 equivalents of water. Such a hydrate may be prepared by crystallizing Compound 1 or 2 of the present invention or a pharmaceutically acceptable salt thereof in water or a water-containing solvent.

In the pharmaceutical composition of the present invention, the active ingredient is preferably Compound 1 represented by formula 1 or a pharmaceutically acceptable salt thereof, and the active ingredient is more preferably tartrate of Compound 1.

In the pharmaceutical composition of the present invention, the active ingredient is preferably sitagliptin or a pharmaceutically acceptable salt thereof and is more preferably sitagliptin phosphate.

In the pharmaceutical composition of the present invention, the active ingredient is preferably vildagliptin or a pharmaceutically acceptable salt thereof.

In the pharmaceutical composition of the present invention, the active ingredient is preferably linagliptin or a pharmaceutically acceptable salt thereof.

The pharmaceutical composition for the prevention or treatment of a non-alcoholic fatty liver disease in accordance with the present invention may be used in the form of a conventional pharmaceutical preparation. That is, upon practical clinical administration, the pharmaceutical composition may be administered in the form of various oral and parenteral dosage forms. In the present invention, oral administration is preferable. Further, a diluent or excipient conventionally known and used in the art, such as a filler, an extender, a binding agent, a wetting agent, a disintegrating agent or a surfactant, may be used upon the formulation of the pharmaceutical composition into a desired dosage form. A solid preparation for oral administration may include a tablet, a pill, a powder, a granule, a capsule, etc., and such a solid preparation is formulated by mixing an active ingredient with at least one excipient such as starch, calcium carbonate, sucrose, lactose, and gelatin. Further, a lubricant such as magnesium stearate or talc may also be used in addition to the excipient.

A liquid preparation for oral administration includes a suspension, a liquid for internal use, an emulsion, a syrup, etc. In addition to a frequently used diluent such as water or liquid paraffin, the liquid preparation may contain a variety of excipients such as a wetting agent, a sweetening agent, an aromatic agent and a preservative. A preparation for parenteral administration includes a sterile aqueous solution, a non-aqueous solution, a suspension, an emulsion, a freeze-dried preparation and a suppository. As a solvent for the non-aqueous solution or suspension, propylene glycol, polyethylene glycol, vegetable oil such as olive oil, injectable ester such as ethyl oleate, or the like may be used. As a base for the suppository, Witepsol, macrogol, Tween 61, cacao butter, laurin butter, glycerogelatin, or the like may be used.

A daily dose or dosage of an active ingredient of the pharmaceutical composition in accordance with the present invention is in the range of 0.1 to 1000 mg/kg, but may vary depending on weight, age, sex, health status and dietary habits of patients, administration times and routes, excretion rates, and severity of disease.

Non-alcoholic fatty liver disease (NAFLD) in the present invention includes both primary and secondary non-alcoholic fatty liver diseases and preferably refers to a non-alcoholic fatty liver disease resulting from primary hyperlipidemia, diabetes or obesity.

Further, the non-alcoholic fatty liver disease (NAFLD) in the present invention includes simple steatosis, non-alcoholic steatohepatitis (NASH), and liver fibrosis and liver cirrhosis occurring due to the progression of these diseases.

The pharmaceutical composition of the present invention may contain at least one active ingredient exhibiting the same or similar function, in addition to the compound of formula 1, 2, 3 or 4 or a pharmaceutically acceptable salt thereof.

Further, the present invention provides a method for the prevention or treatment of a non-alcoholic fatty liver disease, including administering an effective amount of the compound represented by formula 1, 2, 3 or 4 or a pharmaceutically acceptable salt thereof to a mammal (including a human) in need thereof.

As used herein, the term “administration” means the introduction of the pharmaceutical composition of the present invention to a patient via any appropriate method. The pharmaceutical composition of the present invention may be administered via any conventional administration route as long as the pharmaceutical composition can reach a target tissue. For example, the composition may be administered orally, intraperitoneally, intravenously, intramuscularly, subcutaneously, transdermally, intranasally, intrapulmonary, rectally, intracavitally or intrathecally without being limited thereto.

The pharmaceutical composition of the present invention may be administered once a day or may be administered at regular time intervals twice or more a day.

For the prevention and treatment of non-alcoholic fatty liver diseases, the pharmaceutical composition of the present invention may be used alone or in combination with methods employing surgical operation, hormone therapy, medication therapy and biological response modifiers.

Further, the present invention provides use of a compound of formula 1, 2, 3 or 4 or a pharmaceutically acceptable salt thereof for manufacturing a pharmaceutical composition for the prevention and treatment of a non-alcoholic fatty liver disease.

### Advantageous Effects of Invention

The pharmaceutical composition of the present invention has effects of preventing and treating the accumulation of triglyceride (TG) appearing as a typical lesion of non-alcoholic fatty liver, and effects of normalizing alanine aminotransferase (ALT) which is a damage indicator of hepatocytes detected in the blood. Further, the pharmaceutical composition of the present invention inhibits the activation and differentiation of hepatic stellate cells (HSCs) leading to liver fibrosis, thereby suppressing liver fibrosis and furthermore, the progression of liver fibrosis into liver cirrhosis, which provides effects of preventing and treating liver fibrosis or liver cirrhosis. Therefore, the pharmaceutical composition of the present invention may be used as an agent for the prevention or treatment of non-alcoholic fatty liver.

The treatment method of the present invention is useful for the prevention and treatment of non-alcoholic fatty liver diseases.

### BRIEF DESCRIPTION OF DRAWINGS

FIG. 1 shows serum alanine aminotransferase (ALT)-lowering effects of tartrate of Compound 1 or sitagliptin phosphate in non-alcoholic fatty liver-induced mice.

FIG. 2 is a hepatic triglyceride content analysis graph showing hepatic triglyceride-lowering effects of tartrate of Compound 1 or sitagliptin phosphate in non-alcoholic fatty liver-induced mice.

FIG. 3 is a tissue sample photograph showing hepatic triglyceride-lowering effects of tartrate of Compound 1 in non-alcoholic fatty liver-induced mice.

FIG. 4 shows serum ALT-lowering effects of tartrate of Compound 1 in non-alcoholic fatty liver-induced rats.

FIG. 5 is a hepatic triglyceride content analysis graph showing hepatic triglyceride-lowering effects of tartrate of Compound 1 in non-alcoholic fatty liver-induced rats.

FIG. 6 is a tissue sample photograph showing hepatic triglyceride-lowering effects of tartrate of Compound 1 in non-alcoholic fatty liver-induced rats.

FIG. 7 is photograph showing an area of lipid droplets/unit area of a sample through image analysis of a tissue sample exhibiting hepatic triglyceride-lowering effects of tartrate of Compound 1 in non-alcoholic fatty liver-induced rats.

FIG. 8 is a tissue sample image showing hepatic triglyceride accumulation-preventing effects of tartrate of Compound 1, sitagliptin phosphate and vildagliptin on high fat diet-induced fatty liver in mice.

FIG. 9 is an electrophoretic photograph showing target protein expression-lowering effects of tartrate of Compound 1, sitagliptin phosphate, vildagliptin and linagliptin in activated hepatic stellate cells.

FIG. 10 is a graph showing inhibitory effects of tartrate of Compound 1, sitagliptin phosphate and vildagliptin on free fatty acid-induced hepatocytic fat accumulation.

FIG. 11 is an analysis graph showing inhibitory effects of tartrate of Compound 1, sitagliptin phosphate and vildagliptin on TGF-β1 mRNA expression in a CCl₄-induced acute hepatic damage animal model.

### DESCRIPTION OF REFERENCE NUMERALS AND SIGNS

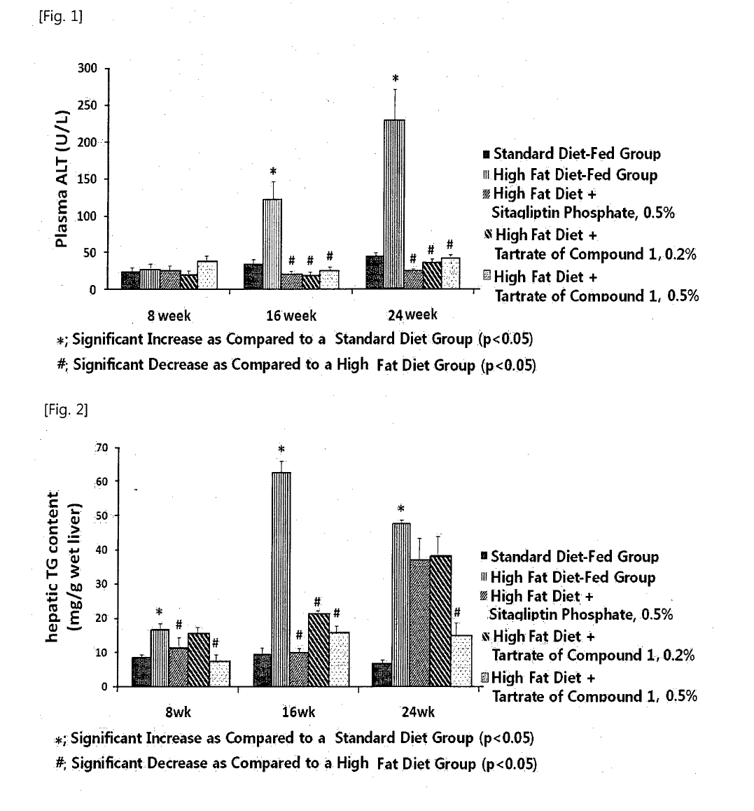

In FIGS. 1, 2, 4, 5 and 7, *: significant increase within the 95% confidence interval (p<0.05), as compared to a standard diet group, and #: significant decrease within the 95% confidence interval (p<0.05), as compared to a high fat diet group.

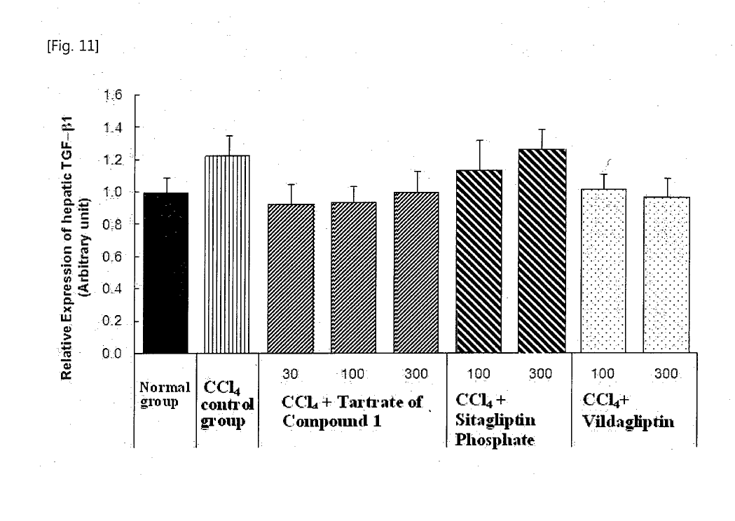

In FIG. 10, *: significant decrease within the 95% confidence interval (p<0.05), as compared to a 0.1% DMSO control group, and **: significant decrease within the 99% confidence interval (P<0.01), as compared to a 0.1% DMSO control group.

### MODE FOR THE INVENTION

Hereinafter, the present invention will be described in more detail with reference to the following Examples. However, the following Examples are provided only for illustrating the present invention and should not be construed as limiting the scope and spirit of the present invention.

### Example 1

### Preventive Effects of Tartrate of Compound 1 and Sitagliptin Phosphate on High Fat Diet-Induced Simple Steatosis in Mice

In order to investigate preventive effects of the tartrate of Compound 1 (prepared according to the method described in Korean Patent Application No. 2008-0036052) and sitagliptin phosphate on simple steatosis, the following experiment was carried out.

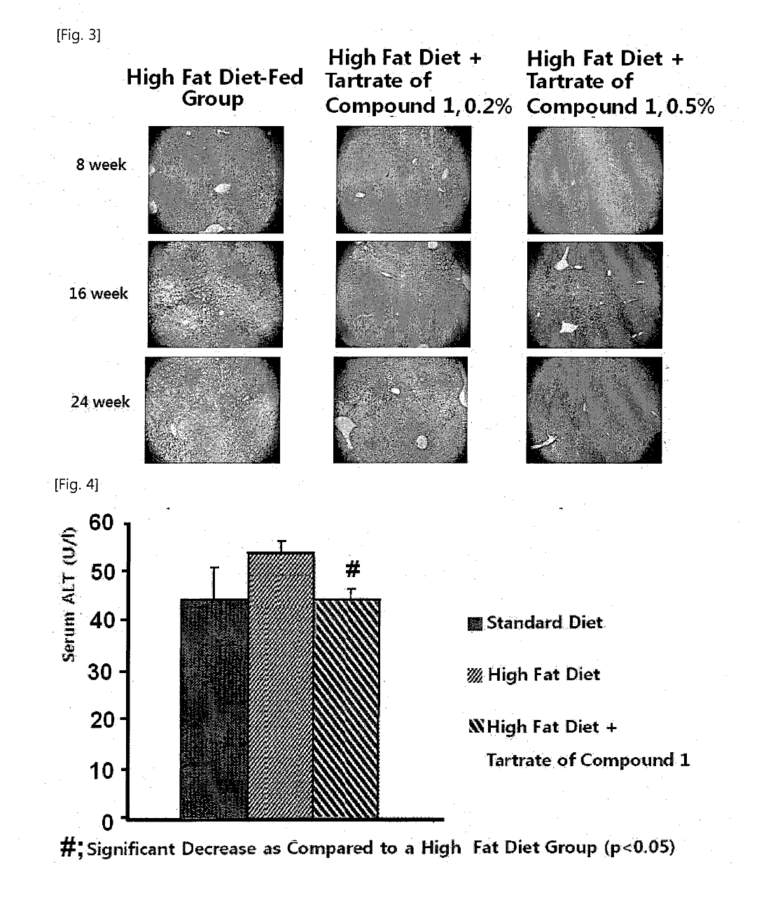

6-week old male C57BL/6 mice were stabilized and then divided into 5 groups. One group was given a standard diet containing 10% fat (trade name: D12450B, manufactured by Research Diets), and one group was given a high fat diet containing 60% fat (trade name: D12492, manufactured by Research Diets). Remaining 3 groups as drug-treated groups were given a drug-mixed diet specially formulated by mixing a high fat diet and a drug. With regard to tartrate of Compound 1, in order to supply 100 mg/kg and 300 mg/kg which are daily target doses of tartrate of Compound 1 based on the daily average high fat diet consumption amount, diets were formulated by mixing a high fat diet and 0.2% by weight and 0.5% by weight of tartrate of Compound 1, respectively. With regard to sitagliptin phosphate, in order to supply 300 mg/kg which is a daily target dose based on the daily average high fat diet consumption amount, a diet was formulated by mixing a high fat diet and about 0.5% by weight of sitagliptin phosphate. Individual drug-treated groups were given the drug-mixed diets thus formulated. 8 weeks, 16 weeks, and 24 weeks after a supply of each diet, animals were dissected and sera were separated. Then, serum ALT levels (alanine aminotransferase, GPT, glutamic pyruvate transaminase) were measured using a blood analyzer (Table 1 and FIG. 1). The excised liver was homogenized in a 5(v/v) % Triton-X solution, and a triglyceride working reagent [which was prepared by mixing a free glycerol reagent (F6428, Sigma) and a triglyceride reagent (T2449, Sigma) in a ratio of 4:1(v/v)] was added thereto, followed by measurement of an absorbance at 540 nm to determine the content of triglyceride (Table 1 and FIG. 2). In addition, in order to observe histological fat distribution, a portion of the excised liver was fixed in a 10(v/v) % formalin solution and then a tissue sample was prepared, followed by hematoxylin & eosin staining and photographing using an image analysis program (computerized image analysis) (FIG. 3, the violet-stained portion represents a normal liver tissue, and white-stained portion represents lipid droplets).

As a result, as shown in FIG. 1, supplies of tartrate of Compound 1 and sitagliptin phosphate exhibited significant decreases in serum alanine aminotransferase (ALT) at 16 weeks and 24 weeks. Further, the hepatic triglyceride content also exhibited a significant decrease in a hepatic triglyceride analysis (FIG. 2) and a histological analysis (FIG. 3), as compared to a high fat diet-fed group. These results represent that tartrate of Compound 1 and sitagliptin phosphate decrease the hepatic triglyceride content against high fat diet-induced fatty liver and therefore have preventive efficacy on fatty liver.

TABLE 1
Fatty liver-preventing effects of tartrate of Compound 1 and sitagliptin
phosphate on high fat diet-induced simple steatosis in mice

| | Plasma ALT (U/L) | | | Hepatic TG (mg/g wet liver) | | |
|---|---|---|---|---|---|---|
| | 8 weeks | 16 weeks | 24 weeks | 8 weeks | 16 weeks | 24 weeks |
| Standard diet-fed group | 25.4 ± 1.4 | 34.0 ± 2.3 | 44.6 ± 5.5 | 8.5 ± 1.0 | 9.4 ± 1.9 | 6.6 ± 1.2 |
| High fat diet-fed group | 27.6 ± 2.5 | 121.1 ± 25.7* | 230.0 ± 41.5* | 16.8 ± 1.7* | 62.4 ± 3.6* | 47.8 ± 1.0* |
| High fat diet + | 20.6 ± 0.9 | 20.5 ± 1.2# | 37.5 ± 4.2# | 15.5 ± 2.0 | 21.5 ± 0.7# | 38.2 ± 5.8 |
| tartrate of | | | | | | |
| Compound 1, 0.2% | | | | | | |
| High fat diet + | 39.3 ± 3.6 | 27.5 ± 1.5# | 43.8 ± 4.3# | 7.4 ± 2.0# | 15.8 ± 2.0# | 15.1 ± 3.5# |
| tartrate of | | | | | | |
| Compound 1, 0.5% | | | | | | |
| High fat diet + | 26.5 ± 1.8 | 22.2 ± 1.3# | 27.0 ± 1.7# | 11.8 ± 2.7# | 10.0 ± 1.2# | 36.8 ± 6.6 |
| sitagliptin | | | | | | |
| phosphate, 0.5% | | | | | | |

*represents a significant increase (p < 0.05), as compared to a standard diet-fed group
# represents a significant decrease (p < 0.05), as compared to a high fat diet-fed group

### Example 2

### Therapeutic Effects of Tartrate of Compound 1 on High Fat Diet-Induced Simple Steatosis in Rats

In order to investigate therapeutic effects of the tartrate of Compound 1 (prepared according to the method described in Korean Patent Application No. 2008-0036052) on simple steatosis, the following experiment was carried out.

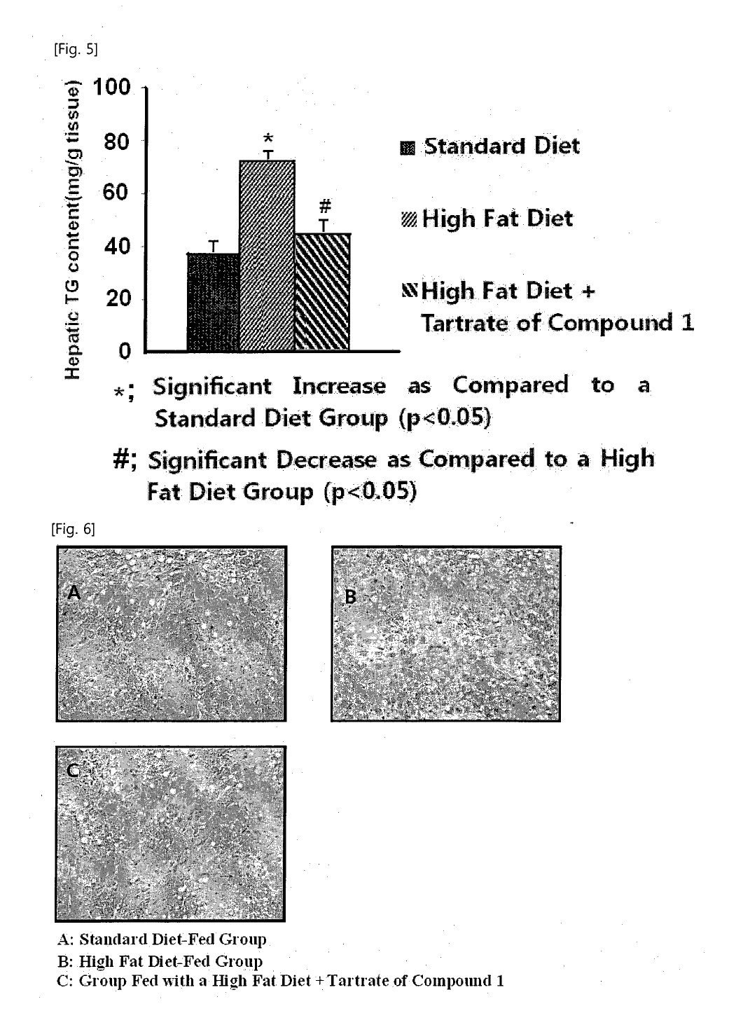

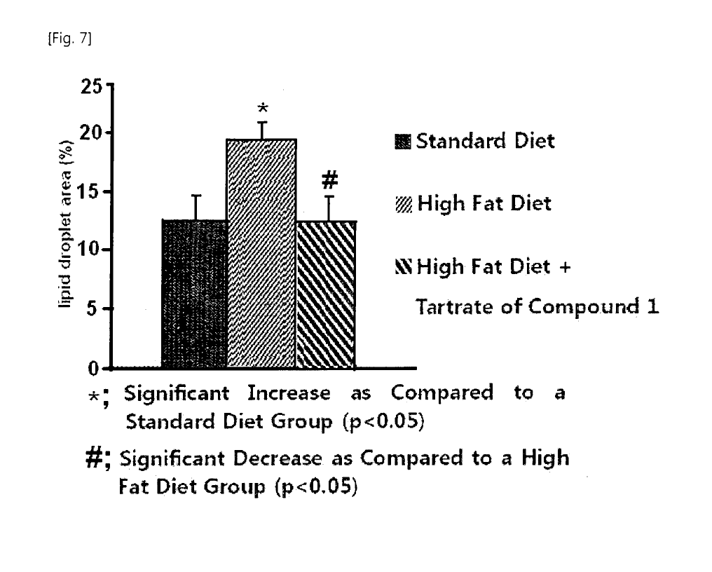

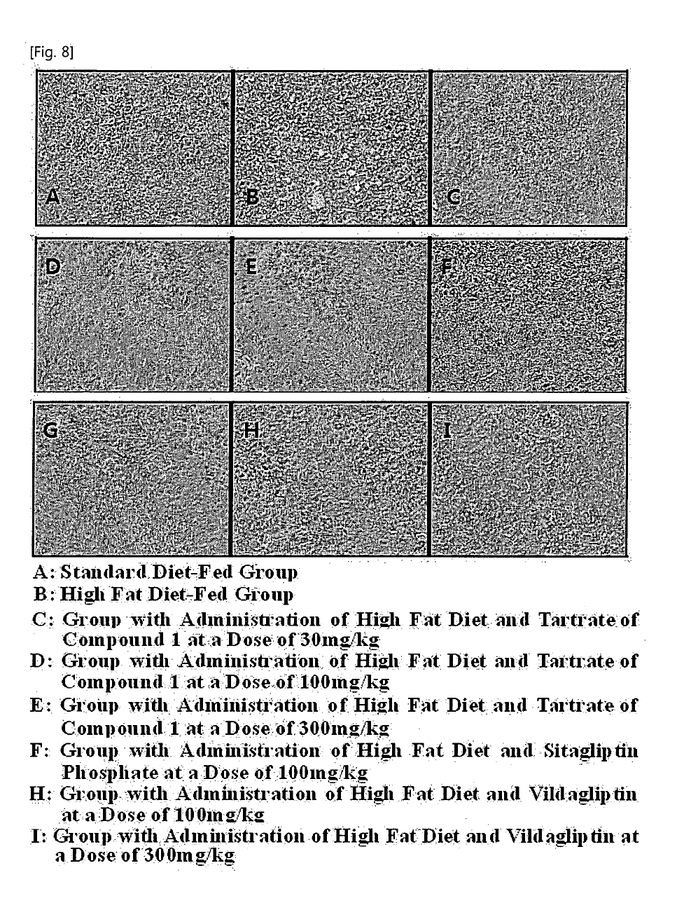

6-week old male rats (Wistar rats) were stabilized and then divided into 2 groups. Animal groups were respectively given a standard diet containing 10% fat (trade name: D12450B, manufactured by Research Diets) and a high fat diet containing 60% fat (trade name: D12492, manufactured by Research Diets) for 24 weeks. When a diet consumption amount was calculated at Week 22 of diet supply, the high fat diet-fed group exhibited a diet intake of 33.40 g/kg. In order to supply 10 mg/kg which is a daily target dose of tartrate of Compound 1, a diet was formulated by mixing a high fat diet and 0.03% by weight of tartrate of Compound 1. 24 weeks after a supply of each diet, animals were divided into a high fat diet-fed group (n=8), a group fed with a high fat diet+0.03% by weight of tartrate of Compound 1 (n=8), and a standard diet-fed group (n=8), followed by feeding for another 14 weeks. Then, animals were dissected and sera were separated. Then, serum ALT levels (alanine aminotransferase), GPT, (glutamic pyruvate transaminase) were measured using a blood analyzer (FIG. 4). The excised liver was homogenized in a 5(v/v) % Triton-X solution, and a triglyceride working reagent [which was prepared by mixing a free glycerol reagent (F6428, Sigma) and a triglyceride reagent (T2449, Sigma) in a ratio of 4:1(v/v)] was added thereto, followed by measurement of an absorbance at 540 nm to determine the content of triglyceride (FIG. 5). In addition, in order to observe histological fat distribution, a portion of the excised liver was fixed in a 10(v/v) % formalin solution and then a tissue sample was prepared, followed by hematoxylin & eosin(HE) staining and photographing using an image analysis program (computerized image analysis) (FIG. 6, the violet-stained portion represents a normal liver tissue, and white portion represents lipid droplets). Thereafter, an area of lipid droplets/unit area of a sample was calculated (FIG. 7).

As a result, as shown in FIG. 4, the administration of tartrate of Compound 1 exhibited a significant decrease in serum alanine aminotransferase (ALT). Further, the hepatic triglyceride content also exhibited a significant decrease in a hepatic triglyceride analysis (FIG. 5) and a histological analysis (FIG. 7), as compared to a high fat diet-fed group. These results represent that tartrate of Compound 1 decreases the hepatic triglyceride content against the existing fatty liver and therefore can be used as an agent for the treatment of fatty liver.

### Example 3

### Non-Alcoholic Fatty Liver-Preventing Effects of Tartrate of Compound 1, Sitagliptin Phosphate and Vildagliptin on High Fat Diet-Induced Simple Steatosis in Mice

In order to investigate preventive effects of the tartrate of Compound 1 on non-alcoholic fatty liver (simple steatosis), the following experiment was carried out.

7-week old male C57BL/6 mice were stabilized and then divided into 9 groups (n=9) according to the body weight and blood glucose level. A standard diet-fed group and a high fat diet-fed group were orally administered a vehicle solution (0.5% MC (methylene cellulose)) of each diet once a day at a dose of 10 ml/kg, and the remaining groups were orally given once a day for 28 days a high fat diet in conjunction with tartrate of Compound 1 at doses of 30, 100 and 300 mg/kg, sitagliptin phosphate at doses of 100 and 300 mg/kg, and vildagliptin at doses of 100 mg/kg and 300 mg/kg, respectively, according to the designated groups. 24 hours after the final oral administration, animals were dissected and the excised liver was homogenized in a 5(v/v) % Triton-X solution to which a triglyceride working reagent was then added, followed by measurement of an absorbance at 540 nm to determine the content of triglyceride (Table 2). In addition, a portion of the excised liver was fixed in a 10(v/v) % formalin solution and then a tissue sample was prepared, followed by hematoxylin & eosin(HE) staining and photographing (FIG. 8).

TABLE 2
Non-alcoholic fatty liver-preventing effects of tartrate of
Compound 1, sitagliptin phosphate and vildagliptin on
high fat diet-induced simple steatosis in mice

| | | Triglyceride | Triglyceride |
|---|---|---|---|
| | | increment | increase |
| | | (%) relative | inhibition* |
| | | to standard | (%) relative |
| Experimental | | diet-fed | to high fat |
| group | Treatment drug and dose | group | diet-fed group |
| Standard diet | 0.5% MC 10 ml/kg | — | — |
| High fat diet | 0.5% MC 10 ml/kg | 54 | — |
| High fat diet + | Tartrate of Compound 1, | 35 | 34 |
| tartrate of | 30 mg/kg | | |
| Compound 1 | Tartrate of Compound 1, | 47 | 12 |
| | 100 mg/kg | | |
| | Tartrate of Compound 1, | 11 | 81 |
| | 300 mg/kg | | |
| High fat diet + | Sitagliptin phosphate | 21 | 61 |
| sitagliptin | 100 mg/kg | | |
| phosphate | Sitagliptin phosphate | 45 | 15 |
| | 300 mg/kg | | |
| High fat diet + | Vildagliptin 100 mg/kg | 38 | 24 |
| vildagliptin | Vildagliptin 300 mg/kg | 11 | 79 |

*% inhibition production increase by taking an increase width of a high fat diet relative to a standard diet to be 100%

As shown in Table 2, when compared with the standard diet-fed group, the high fat diet-fed group exhibited a 54% increase in the hepatic triglyceride content; the group with administration of tartrate of Compound 1 at a dose of 300 mg/kg exhibited an 11% increase in the hepatic triglyceride content; the group with administration of sitagliptin phosphate at a dose of

100 mg/kg exhibited a 21% increase in the hepatic triglyceride content; and the group with administration of vildagliptin at a dose of 300 mg/kg exhibited an 11% increase in the hepatic triglyceride content, thus demonstrating that compounds of the present invention have preventive effects against the occurrence of high fat diet-induced fatty liver.

Further, the maximum decrement of the hepatic triglyceride content relative to the high fat diet-fed group was a maximum of 81% in the group with administration of tartrate of Compound 1; a maximum of 61% in the group with administration of sitagliptin phosphate; and a maximum of 79% in the group with administration of vildagliptin, also thus demonstrating that compounds of the present invention exhibit preventive efficacy against the occurrence of fatty liver. When a tissue sample was photographed for the histological evaluation of this preventive efficacy, a reduction of lipid droplets by tartrate of Compound 1, sitagliptin phosphate, and vildagliptin was confirmed (FIG. 8). These results suggest that all of these three drugs have preventive efficacy against the occurrence of fatty liver.

### Example 4

### Inhibitory Effects of Tartrate of Compound 1, Sitagliptin Phosphate, Vildagliptin and Linagliptin on Activation of Rat Hepatic Stellate Cells

An inhibitory action of the compounds of the present invention on the activation of hepatic stellate cells was tested according to the following cell culture method. Male rats (Wistar rats), weighing 500 to 700 g, were anesthetized, followed by an abdominal incision, and cannulae were connected to hepatic portal veins, followed by sequential perfusion with a Hank's Balanced Salt Solution (HBSS) containing heparin and an HBSS containing type 1 collagenase. After the perfusion was complete, the liver was excised, ground with surgical scissors, and added to an HBSS containing type 1 collagenase, followed by shaking culture at 37° C. for 15 minutes.

The completely ground liquid-state liver tissue was passed through gauze and centrifuged at 500 g for 10 minutes. The resulting mingled cell precipitate was washed with a phosphate buffer, followed by centrifugation at 100 g for 5 minutes, and the supernatant was collected. The supernatant was further centrifuged at 500 g for 10 minutes to obtain a precipitate to which a 9:1 (v/v) mixture of a Ficoll liquid (GE Healthcare) and a Percoll liquid (GE Healthcare) was added, followed by mixing. A phosphate buffer was gently placed on the mixed layer, followed by centrifugation at 1,400 g for 15 minutes. Then, the cell layer formed between the upper layer and the lower layer was recovered and washed once with a Dulbecco's Modified Eagle Medium (DMEM) containing 10(v/v) % fetal bovine serum. The resulting cells were added at a cell density of 3.1×10⁵ cells/cm², the medium was exchanged with a fresh medium after 24 hours, and cultured for 7 days with exchange of the medium at intervals of 2 to 3 days. Then, the drugs were treated at specified concentrations in a medium containing 1 ng/ml of a human transforming growth factor (TGF) β1 and a medium containing no TGF β1. TGF β1 was dissolved in dimethylsulfoxide (DMSO) to a concentration 1.000-fold higher than the desired treatment concentration of the drug, 1.000-fold diluted in a medium to be a 1-fold (1×) volume, and then treated on cells.

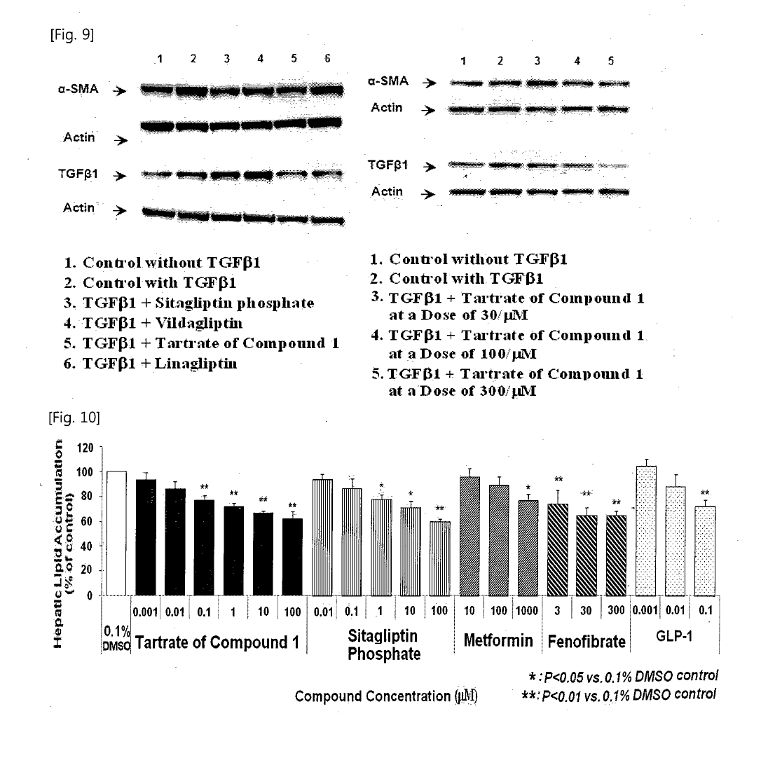

After 7 days of the drug treatment, the medium was harvested and washed with a phosphate buffer. A buffer containing a surfactant was added thereto and the cells were lysed. Then, proteins were quantified, electrophoresed on a 4-12% Bis-Tris gel (Invitrogen), and transferred to a nitrocellulose membrane. The transferred proteins were reacted with primary antibodies specific for α-smooth muscle actin (α-SMA) or TGFβ1, and then reacted with horse radish peroxidase-conjugated specific secondary antibodies. The expression level of the target protein was confirmed using a chemiluminescent liquid and corrected in terms of actin (β-actin) expression level (FIG. 9). The decrement of observed protein expression by 100 μM of individual compounds of the present invention was expressed as a percentage drug-induced decrement, based an increment of protein expression in the TGFβ1-treated positive control group relative to the negative control group to which only DMSO was added (Table 3).

TABLE 3
Expression-lowering effects on activity indicator proteins in activated
hepatic stellate cells

| | 1 ng/mL TGFβ1-induced protein | |
|---|---|---|
| | expression inhibition rate (N = 3) | |
| | TGFβ1 | α-SMA |
| Sitagliptin phosphate | −6 ± 51.9% | 46 ± 7.3% |
| Vildagliptin | 14 ± 32.8% | −56 ± 8.4% |
| Tartrate of Compound 1 | 78 ± 17.2% | 25 ± 1.9% |
| Linagliptin | 64 ± 17.2% | 159 ± 17.5% |

As a result, as shown in Table 3 and FIG. 9, it was confirmed that protein expression levels of TGFβ1 and α-SMA, which are important factors for the pathophysiology of liver fibrosis increased due to hTGF-β1 in activated hepatic stellate cells of rats, were lowered by tartrate of Compound 1, sitagliptin phosphate, vildagliptin, and linagliptin, and it was further confirmed that a protein expression decrement of TGFβ1 and α-SMA is increased with a higher concentration of tartrate of Compound 1 (FIG. 9). These results suggest that tartrate of Compound 1, sitagliptin phosphate, vildagliptin, and linagliptin are capable of exhibiting therapeutic effects on steatohepatitis and liver fibrosis.

### Example 5

### Inhibitory Effects of Tartrate of Compound 1 and Sitagliptin Phosphate on Free Fatty Acid-Induced Intracellular Fat Accumulation in Human Hepatoma Cells

Treatment of hepatocytes with free fatty acid results in an increase in intracellular fat accumulation. By a combined treatment of free fatty acid and a drug, inhibitory effects of individual drugs on intracellular fat accumulation were quantified using a triglyceride staining method.

The human hepatoma cell line, HepG2 cells were cultured in a minimum essential medium (MEM) containing 10(v/v) % fetal bovine serum for 48 hours. Then, the culture medium was exchanged with a 0.5 mM free fatty acid mixture made by dissolving oleate and palmitate (Sigma) in a molar ratio of 2:1 in a MEM containing 1(v/v) % bovine serum albumin, and 0.1(v/v) % dimethyl sulfoxide (DMSO) or each test drug was added thereto, followed by cell culture for 24 hours. For the negative control group, a MEM containing 1(v/v) % bovine serum albumin was treated with 0.1(v/v) % dimethyl sulfoxide with no addition of free fatty acid. After the culture was complete, the medium was removed and the cells were washed with a phosphate buffer. An undiluted solution of 10 mM Nile Red (Sigma) dissolved in dimethyl sulfoxide containing 1(v/v) % Pluronic F127 (Invitrogen) was 1.000-fold diluted in a phosphate buffer and added to the cells, followed by staining of intracellular fat at 37° C. and 200 rpm under light-shielded conditions for 30 minutes. After the staining was complete, the supernatant was discarded and replaced with a phosphate buffer, followed by measurement of fluorescence intensities under conditions of a 488 nm excitation wavelength and a 550 nm emission wavelength. Then, in order to correct a deviation according to the cell count, a phosphate buffer containing 10 μM resazurin (Sigma) dissolved therein was added to the same well. Thereafter, before and after the reaction at 37° C. for 1 hour under light-shielded conditions, fluorescence intensities were measured using a fluorometer under conditions of a 535 nm excitation wavelength and a 580 nm emission wavelength, whereby an increase in fluorescence intensity due to resorufin reduced and formed by the intracellular mitochondrial activity was measured. Using the value of a fluorescence intensity of Nile Red corrected in terms of an increase in fluorescence intensity due to the reduction of resazurin, the experimental results were expressed as a percentage for the fat accumulation increased by a treatment with free fatty acid, as compared to the negative control group.

As a result, as shown in FIG. 10, tartrate of Compound 1 and sitagliptin phosphate exhibited dose-dependent inhibition of free fatty acid-induced fat accumulation in hepatocytes. 28.7±3.2% inhibitory effects thereof exhibited at a dose of 1 μM are equivalent to 28.0±4.9% inhibitory effects of GLP-1 (Ding X, et al., Hepatology 2006, 43:173-181; Gupta N A, et al., Hepatology 2010, 51(5):1584-1592), which is known to inhibit the fatty acid biosynthesis through the activation of a glucagon-like peptide-1 (GLP-1) receptor of the hepatocyte membrane, exhibited at a dose of 100 nM, 35.6±6.5% inhibitory effects of fenofibrate (Hahn S E & Goldgerg D M, Biochem Pharmacol 1992, 43(3):625-33), which is known to decrease fat accumulation through the promotion of fatty acid oxidation, exhibited at a dose of 30 μM, and 23.6±5.0% inhibitory effects of metformin (Zang M et al., J Biol Chem, 2004, 279(46):47898-47905), which is reported to promote fatty acid oxidation and inhibit fatty acid biosynthesis through the activation of AMPK-activated protein kinase (AMPK), exhibited at a dose of 1 mM. These results suggest that tartrate of Compound 1 and sitagliptin phosphate are capable of exhibiting fatty liver-preventing effects by direct action on hepatocytes to inhibit fat accumulation, in conjunction with indirect effects due to an increased level of endogenous GLP-1.

### Example 6

### Inhibitory Effects of Tartrate of Compound 1, Sitagliptin Phosphate and Vildagliptin on Activation of TGF-β1 in CCl₄-Induced Acute Hepatic Damage Animal Model of Mice

In order to investigate inhibitory effects of compounds of the present invention on expression of TGF-β1 which is known to play a crucial role in a fibrosis process of hepatocytes in a CCl₄-induced acute hepatic damage mouse model, the following experiment was carried out. 7-week old male C57BL/6 mice were stabilized and divided into 9 groups (n=7) according to the body weight, followed by intraperitoneal administration of CCl₄ (0.1 ml/kg). The normal control group was administered an olive oil. The control group was orally administered a vehicle solution (0.5% MC) once a day at a dose of 10 ml/kg, at intervals of 24 hours for 3 days. The remaining groups were orally administered once a day tartrate of Compound 1 at doses of 30, 100 and 300 mg/kg, sitagliptin phosphate (prepared according to the method described in WO2004/085378 or WO2005/003135) at doses of 100 and 300 mg/kg, and vildagliptin (Trademax, China) at doses of 100 and 300 mg/kg, respectively, according to the designated groups. 1 hour after the final oral administration, animals were dissected and the excised liver was subjected to evaluation of TGF-β1 mRNA expression. The liver tissue stored in liquid nitrogen was homogenized in a TRIZOL solution and then the total RNAs were extracted. The isolated RNAs were diluted to a concentration of 1 μg/μL in 0.1(v/v) % DEPC (diethyl pyrocarbonate), and then the RNA mixture [2 μg/μL each RNA+2 μL of 0.5 μg/μL oligo d(T)15+up to 15 μL of 0.1% DEPC water] was incubated using a PCR apparatus at 72° C. for 5 minutes, followed by quenching on ice. The mixture [1 μL of PCR nucleotide mix+5 μL of 5×MMLV RT bf.(Promega, M531A)+1 μL of 200 μg/μL M-MLV reverse] was prepared, and then subjected to elongation at 42° C. for 60 minutes, denaturation at 95° C. for 5 minutes and quenching at 8° C. to synthesize cDNAs which were stored at −20° C. until subsequent use. In order to carry out RT-PCR, the mixture [1 μL of 20 pmol each Primer+9.5 μL of Nuclease-free water] was prepared and then 2 μL of each cDNA was placed in sterile PCR tubes to which 12.5 μL of remix Taq™ (RR003A, TaKaRa) was then added, followed by mixing. PCR was carried out under the following conditions (Table 4), using Thermal Cycler (PTC-200, MJ Research). PCT products were electrophoresed and analyzed using an Image Analyzer (Vilber Lourmat). Using β-actin expression, the expression of a target gene TGF-β1 was subjected to normalization (FIG. 11). The results are given in Table 5.

TABLE 4
RT-PCR test conditions and PCR primer sequence

| | Temperature | Duration | Cycle No. |
|---|---|---|---|
| Pre-Denaturation | 94° C. | 5 min | |
| Denaturation | 94° C. | 30 sec | |
| Annealing | 57° C. for mβActin | 60 sec | 25 N |
| | 62° C. for mTGF-β1 | 30 sec | 32 N |
| Elongation | 72° C. | 60 sec | |
| Post-Elongation | 72° C. | 7 min | |
| Post-Cooling | 8° C. | | |

PCR primer

| | |
|---|---|
| mTGFβ1 F1 | 5′-tgc gct tgc aga gat taa aa-3′ (20) |
| mTGFβ1 R1 | 5′-ctg ccg tac aac tcc agt ga-3′ (20) |
| | 156 bp |
| mβActin F | 5′-cca gag caa gag agg tat cc-3′ (20) |
| mβActin R | 5′-agg tct tta cgg atg tca acg-3′ (21) |
| | 704 bp |

TABLE 5
Inhibitory effects of tartrate of Compound 1, sitagliptin phosphate and
vildagliptin on activation of TGF-β1 in CCl₄-induced acute hepatic
damage mouse model

| | | | TGF-β1 |
|---|---|---|---|
| | | | expression |
| | | | inhibition |
| | | TGF-β1 | rate* (% |
| | | expression | CCl₄ |
| Experimental | | (normalized | control |
| group | Treatment drug and dose | by actin) | group) |
| Normal group | 0.5% MC | 1.00 ± 0.10 | |
| CCl₄ control group | 0.5% MC | 1.22 ± 0.13 | — |
| CCl₄ + tartrate of | Tartrate of Compound 1, | 0.92 ± 0.13 | 135 |
| Compound 1 | 30 mg/kg | | |
| | Tartrate of Compound 1, | 0.93 ± 0.10 | 128 |
| | 100 mg/kg | | |
| | Tartrate of Compound 1, | 1.00 ± 0.13 | 100 |
| | 300 mg/kg | | |
| CCl₄ + sitagliptin | Sitagliptin phosphate | 1.13 ± 0.19 | 41 |
| phosphate | 100 mg/kg | | |
| | Sitagliptin phosphate | 1.26 ± 0.13 | −17 |
| | 300 mg/kg | | |
| CCl₄ + vildagliptin | Vildagliptin 100 mg/kg | 1.02 ± 0.09 | 92 |
| | Vildagliptin 300 mg/kg | 0.96 ± 0.12 | 115 |

*% inhibition production increase by taking an increase width of a CCl₄ control group relative to a normal group to be 100%

As a result, as shown in Table 5 and FIG. 11, the CCl₄ control group exhibited a 22% increase in expression of mRNA of TGF-β1 of the liver, as compared to the normal group, and the expression of TGF-β1 was subjected to normalization in terms of tartrate of Compound 1 and vildagliptin. The sitagliptin phosphate-treated group also exhibited decreased expression of TGF-β1 as compared to the CCl₄ group, thus showing inhibitory effects on CCl₄-induced TGF-β1 expression. Further, a decrement in TGF-β1 mRNA expression of the liver relative to the CCl₄ control group was a maximum of 135% in the group with administration of tartrate of Compound 1, a maximum of 41% in the group with administration of sitagliptin, and a maximum of 115% in the group with administration of vildagliptin, thus suggesting that the compounds of the present invention have medicinal efficacy against the occurrence of liver fibrosis due to increased expression of TGF-β1.

### Example 7

### Alanine Aminotransferase-Lowering Effects of Tartrate of Compound 1 and Sitagliptin Phosphate in Obesity Animal Model ob/ob Mice

In order to investigate therapeutic effects of tartrate of Compound 1 and sitagliptin phosphate on simple steatosis, the following experiment was carried out.

5-week old male C57BL/6 mice (normal mouse group) and ob/ob mice (obesity mouse group) were stabilized and then divided into 6 groups according to the body weight and blood glucose level, followed by feeding with a standard diet and a drug-mixed diet. The diet consumption amount of mice exhibits a diet intake of about 1 g/10 g of mouse body weight. Therefore, in order to supply 10, 100 and 300 mg/kg which are daily target doses of tartrate of Compound 1, diets were formulated by mixing a standard diet with 0.01% by weight, 0.1% by weight and 0.3% by weight of tartrate of Compound 1 and 0.3% by weight of sitagliptin phosphate, respectively. 4 weeks after a supply of diets, animals were dissected and sera were separated. Plasma alanine aminotransferase levels were measured using a blood analyzer (Table 6).

TABLE 6
Plasma alanine aminotransferase (ALT)-lowering
effects of tartrate of Compound 1 and sitagliptin phosphate
in obesity animal model ob/ob mice

| | | ALT | ALT increase |
|---|---|---|---|
| | | increment | inhibition* |
| | | (%) relative | (%) relative |
| Experimental | | to normal | to ob/ob mouse |
| group | Treatment drug and dose | mouse group | control group |
| Normal mouse | Standard diet | — | — |
| group | | | |
| ob/ob mouse | Standard diet | 1001 | — |
| control group | | | |
| Tartrate of | Mixture of standard diet | | |
| Compound 1 | with tartrate of | 610 | 43 |
| | Compound 1, 0.01% | | |
| | Mixture of standard diet | | |
| | with tartrate of | 653 | 39 |
| | Compound 1, 0.1% | | |
| | Mixture of standard diet | | |
| | with tartrate of | 757 | 27 |
| | Compound 1, 0.3% | | |
| Sitagliptin | Mixture of standard diet | | |
| phosphate | with sitagliptin | 869 | 15 |
| | phosphate 0.3% | | |

*% inhibition production increase by taking an increase width of a high fat diet relative to a standard diet to be 100%

As a result, it was confirmed that the administration of tartrate of Compound 1 or sitagliptin phosphate results in a reduction of plasma alanine aminotransferase. These results suggest that tartrate of Compound 1 and sitagliptin phosphate exhibit a decreasing action on alanine aminotransferase increased due to fatty liver and therefore can be used as a therapeutic agent for the treatment of simple steatosis.

1. A pharmaceutical composition for the prevention or treatment of a non-alcoholic fatty liver disease, comprising an active ingredient selected from among Compound 1 represented by formula 1, sitagliptin, vildagliptin, linagliptin and pharmaceutically acceptable salts thereof.

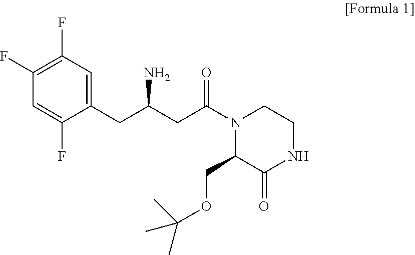 [SMILES: CC(C)(C)OC[C@@H]1C(=O)NCCN1C(=O)C[C@H](N)Cc1cc(F)c(F)cc1F]

2. The composition according to claim 1, wherein the active ingredient is Compound 1 represented by formula 1 or a pharmaceutically acceptable salt thereof.

3. The composition according to claim 2, wherein the active ingredient is tartrate of Compound 1.

4. The composition according to claim 1, wherein the active ingredient is sitagliptin or a pharmaceutically acceptable salt thereof.

5. The composition according to claim 4, wherein the active ingredient is sitagliptin phosphate.

6. The composition according to claim 1, wherein the active ingredient is vildagliptin or a pharmaceutically acceptable salt thereof.

7. The composition according to claim 1, wherein the active ingredient is linagliptin or a pharmaceutically acceptable salt thereof.

8. The composition according to any one of claims 1 to 7, wherein the non-alcoholic fatty liver disease results from hyperlipidemia, diabetes or obesity.

9. The composition according to any one of claims 1 to 7, wherein the non-alcoholic fatty liver disease is selected from the group consisting of simple steatosis, non-alcoholic steatohepatitis, liver fibrosis and liver cirrhosis.

10. A method for the prevention or treatment of a non-alcoholic fatty liver disease, comprising administering an effective amount of an active ingredient selected from among Compound 1 represented by formula 1, sitagliptin, vildagliptin, linagliptin and pharmaceutically acceptable salts thereof to a mammal including a human in need thereof.

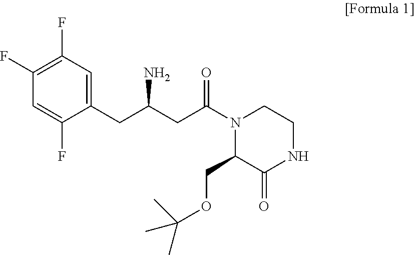 [SMILES: CC(C)(C)OC[C@@H]1C(=O)NCCN1C(=O)C[C@H](N)Cc1cc(F)c(F)cc1F]

11. The method according to claim 10, wherein the active ingredient is Compound 1 represented by formula 1 or a pharmaceutically acceptable salt thereof.

12. The method according to claim 11, wherein the active ingredient is tartrate of Compound 1.

13. The method according to claim 10, wherein the active ingredient is sitagliptin or a pharmaceutically acceptable salt thereof.

14. The method according to claim 13, wherein the active ingredient is sitagliptin phosphate.

15. The method according to claim 10, wherein the active ingredient is vildagliptin or a pharmaceutically acceptable salt thereof.

16. The method according to claim 10, wherein the active ingredient is linagliptin or a pharmaceutically acceptable salt thereof.

17. The method according to any one of claims 10 to 16, wherein the non-alcoholic fatty liver disease results from hyperlipidemia, diabetes or obesity.

18. The method according to any one of claims 10 to 16, wherein the non-alcoholic fatty liver disease is selected from the group consisting of simple steatosis, non-alcoholic steatohepatitis, liver fibrosis and liver cirrhosis.

19.-27. (canceled)


SEQUENCE LISTING (4 sequences)

SEQ ID NO: 1 - DNA, 20 bp, Artificial Sequence
  mTGFbeta F1 primer
tgcgcttgcagagattaaaa

SEQ ID NO: 2 - DNA, 20 bp, Artificial Sequence
  mTGFbeta R1 primer
ctgccgtacaactccagtga

SEQ ID NO: 3 - DNA, 20 bp, Artificial Sequence
  mbeta Actin F primer
ccagagcaagagaggtatcc

SEQ ID NO: 4 - DNA, 21 bp, Artificial Sequence
  mbeta Actin R primer
aggtctttacggatgtcaacg